(19) United States
(12) Patent Application Publication
(10) Pub. No.: US 2004/0260350 A1
(43) Pub. Date: Dec. 23, 2004

(54) Automatic EGM amplitude measurements during tachyarrhythmia episodes

(75) Inventors: John S. Brandstetter, Coon Rapids, MN (US); Vasant Padmanabhan, Maple Grove, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/600,881

(22) Filed: Jun. 20, 2003

(51) Int. Cl. A61N 1/362

(52) U.S. Cl. 607/14

(57) ABSTRACT

In an IMD, when tachyarrhythmia detection criterion are satisfied or a high intrinsic heart rate is detected, a peak amplitude detection circuit is enabled to detect the peak amplitude of the cardiac signal of interest, i.e., the P-wave in the case of atrial tachyarrhythmias and the R-wave in case of ventricular tachyarrhythmias. Peak amplitude data is accumulated for subsequent use in setting sensing thresholds and/or gain of a sense amplifier to reduce undersensing of lower amplitude cardiac signals during such tachyarrhythmias.

### FIELD OF THE INVENTION

This invention relates to implantable medical devices (IMDs), and more particularly to improved methods and apparatus for measuring amplitudes of EGM signals of interest during tachyarrhythmia episodes to provide data enabling optimal programming of sensing thresholds.

### BACKGROUND OF THE INVENTION

By way of definition, the term “tachyarrhythmia” refers to fast, abnormal rhythms of a heart chamber that include and atrial flutter or fibrillation (AF) and ventricular flutter or fibrillation (VF), which may be amenable of conversion to a normal sinus rhythm (NSR) by “cardioversion” or “defibrillation”, and tachycardias that may be amenable to conversion to NSR by the application of certain ATP regimens to the affected heart chamber. Such tachycardias include atrial tachycardia (AT) originating from one or more ectopic site in the atria, ventricular tachycardia (VT) originating from one or more ectopic site in the ventricles, and supraventricular tachycardia (SVT) resulting from high rate atrial depolarizations conducted to the ventricles, atrial tachycardia (AT). Individuals whose heart's go from sinus rhythm into high rate, non-sinus, AT and VT episodes can suffer debilitating physiologic effects that in certain cases can progress to more dangerous high rate VT or VF that lead to sudden cardiac death (SCD) unless the ventricles are cardioverted or defibrillated within a very short time after onset of such high rate VT or VF.

High rate, non-sinus, AT and VT episodes can often be terminated by the application of respective atrial and ventricular anti-tachyarrhythmia pacing (ATP) regimens that comprise single overdrive pacing pulses or bursts of a plurality of high rate pacing pulses. However, there is a danger that such ATP regimens may not be effective. Consequently, automatic implantable arrhythmia control devices, particularly implantable cardioverter/defibrillators (ICDs) that provide staged or tiered therapies including such ATP regimens and cardioversion/defibrillation regimens (also referred to as pacemaker/cardioverter/defibrillators or PCDs), are implanted in patients that are susceptible to this risk. Among the most important functions of such ICDs are to sense cardiac signals comprising the P-wave and/or R-wave, to detect and correctly identify the particular tachyarrhythmia that is occurring as a function of meeting specific detection criteria, to supply an appropriate cardioversion/defibrillation or ATP burst pacing regimen, and to determine whether or not the supplied regimen was effective. Current ICOs employ tachyarrhythmia classification algorithms that generally characterize heart rates between 150 and 250 bpm as tachycardias that can be further differentiated by their EGM configuration as either monomorphic or polymorphic. Atrial or ventricular arrhythmias exhibiting heart rates above the upper AT or VT range are typically classified as AF or VF, respectively.

The implantable pulse generators (IPGs) of bradycardia and ATP pacemakers and ICDs as well as implantable cardiac monitors include sense amplifiers that function to detect certain characteristics of the electrogram (EGM) of the heart, typically the P-waves of atrial contractions and/or R-waves of ventricular contractions. Such sense amplifiers are coupled to sense electrodes, typically located in operative relation to the atria and/or ventricles, through electrical medical lead conductors that conduct the cardiac signals to the sense amplifier input terminals. In certain implantable cardiac monitors that do not include electrical medical leads, the sense electrodes are located on the housing of the implantable cardiac monitor.

The atrial or ventricular EGM signal conducted to the sense amplifier input terminals is typically amplified by a programmable gain sense amplifier stage, and the amplified signal is compared to a programmable sense or sensing threshold. The sense amplifier is therefore characterized as having a programmable gain and sensitivity or sensing threshold. An atrial or ventricular sense event is generated by the sense amplifier when the respective amplified atrial or ventricular EGM signal amplitude exceeds the sensing threshold. The atrial or ventricular sense event signals are employed in algorithms or circuitry for detecting the above-described dangerous cardiac arrhythmias of the heart chamber, including atrial or ventricular bradycardia, AT, AF, VT, and VF, typically as a function of a measured time interval between consecutive sense event signals.

Typically, the sensing threshold or sensing threshold is programmed to a value determined by the attending physician after careful study of the variety of P-wave, R-wave and T-wave amplitudes in the EGM experienced by a patient that is typically experiencing bradycardia or in NSR during the study. The physician attempts to set the sensing threshold and sense amplifier gain of the atrial or ventricular sense amplifier so that it does not fail to sense or “undersense” the P-waves or R-waves and avoids “oversensing” of noise transients or undesirable signals, e.g., far field EGM signals or T-wave signals, giving rise to erroneous sensing of cardiac events. After the sensing threshold or an atrial or ventricular sense amplifier is programmed, any cardiac signal EGM amplitude exceeding the programmed sensing threshold is characterized as an atrial or ventricular sense event, respectively.

The physician must schedule the follow-up visits with the patient and expend the time necessary to conduct to test and properly adjust the sensing threshold having an appropriate sensing margin that is expected to minimize oversensing and undersensing. However, the physician generally sees the patient only occasionally, and weeks or months may go by without the sensing threshold being changed. The sensing threshold that will accurately detect P-waves or R-waves does not stay static. The P-wave and R-wave amplitudes and frequency content can vary considerably over time due to a number of factors including physical and mental stress, certain transient or chronic illnesses, and changes in the electrode-tissue interface due to foreign body reactions or sense electrode movement. Moreover, the delivery of atrial and/or ventricular pacing pulses causes evoked response P-waves and/or R-waves having higher amplitudes than intrinsic P-waves and/or R-waves.

Consequently, systems and algorithms for automatically varying the respective atrial or ventricular sense amplifier gain and/or sensing thresholds within the cardiac cycle or cycles following detection of a respective intrinsic P-wave or R-wave or delivery of a respective atrial or ventricular pacing pulse been implemented. Such systems and algorithms include those disclosed in U.S. Pat. Nos. 5,117,824, 5,269,300, 5,339,820, 5,370,124, 5,560,369, 5,620,466, 5,658,317, 5,662,688, and 5,913,880, for example. These approaches still require the physician to determine and program a base sensing threshold that is automatically varied depending upon the particular algorithm to avoid sensing the evoked response and/or the T-wave following delivery of a ventricular pacing pulse

External cardiac monitors and most IMDs of the above-described types store data relating to sensed events, the detection of arrhythmias, short segments of the ECG or EGM, typically accumulated when arrhythmia detection criteria are met, and the therapies (if any) that are delivered. Exemplary, data accumulation systems and algorithms are disclosed in U.S. Pat. Nos. 4,250,888, 4,360,030, 4,513,743, 5,313,953, 5,330,513, and 5,331,966, for example. In certain instances, rate histograms are developed and stored in memory from the monitored intrinsic intervals measured between successive ventricular and/or atrial sense events.

In MEDTRONIC® Marquis® Model 7230 and 7274 ICD IPGs, for example, cardiac data is stored in IPG memory until it is subsequently uplink telemetry (UT) transmitted to an external medical device (EMD), e.g., a programmer, that displays or prints it out for analysis by the physician. The R-wave peak amplitude is detected using the R-wave sense amplifier, digitized, and processed to provide R-wave peak trend data and the most recent R-wave peak amplitude while bradycardia pacing is delivered, if necessary, prior to detection of a VT or VF that are stored in the IPG memory. Such R-wave peak amplitude data storage is halted when the VT or VF detection criteria are met, and a separate wide band EGM amplifier is enabled to record the EGM sensed between a selected sense electrode pair during the VT or VF episode prior to and following delivery of an anti-tachyarrhythmia therapy. During a later telemetry session, wide band EGM strip data are UT transmitted to the external programmer to be reconstituted and displayed or printed in the manner of an EGM tracing. The accumulated R-wave peak amplitude trend and most recent actual R-wave peak amplitude are also UT transmitted to the external programmer to be reconstituted and displayed or printed in a graphical manner. Such R-wave peak data and wide band EGM does not necessarily lend itself to being readily used to program the ventricular sense amplifier sensing threshold and/or gain to reliably sense R-waves during a VT or VF episode.

A need remains for an effective way of providing data that can be used by the physician to program the sensing threshold and/or gain of sense amplifiers to improve sensing of cardiac signals of interest during tachyarrhythmia episodes when the cardiac signal amplitudes tend to lessen.

### BRIEF SUMMARY OF THE INVENTION

In accordance with the present invention, peak amplitude data of the cardiac signal of interest, i.e., the P-wave in the case of atrial tachyarrhythmias and the R-wave in case of ventricular tachyarrhythmias, is accumulated during tachyarrhythmia episodes. The accumulated data can be employed for diagnostic purposes, e.g., for studying the affects of drug treatments or ICD therapies or for setting sensing thresholds and/or gain of a sense amplifier to reduce undersensing of lower amplitude cardiac signals during such tachyarrhythmias.

In accordance with a preferred embodiment of the invention, when tachyarrhythmia detection criteria are satisfied or a high intrinsic heart rate is detected or other pre-detection criterion are met, a peak amplitude detection circuit is enabled to detect the cardiac signal peak amplitude. The peak amplitudes of a succession of intrinsic cardiac signals associated with the tachyarrhythmia episode are detected and stored in IMD memory, optionally time and date stamped and associated with other physiologic data and data related to the tachyarrhythmia episode and delivery of any anti-tachyarrhythmia therapies. The accumulated episode data can be stored until the IMD memory is subsequently UT transmitted to an external medical device (EMD), e.g., a programmer. The data can then be analyzed by the physician to set a suitable sensing threshold for the sense amplifier that is normally used in monitoring cardiac activity.

Advantageously, the UT transmitted peak amplitude data can be more readily used to program (or automatically set) the sensing threshold or gain of the sense amplifier because the measurements are made during a tachyarrhythmia episode. Proper operation of the sensing circuitry in a tachyarrhythmia monitor or therapy delivery IMD requires appropriate detection of arrhythmic events that could be at lower amplitudes than non-arrhythmic events.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic illustration of an ICD IPG and associated ICD leads extending from the ICD IPG to cardioversion/defibrillation and pace/sense electrodes located in operative relation to the ventricles of a heart;

FIG. 2 is a schematic block diagram of the circuitry of the ICD IPG of FIG. 1 in which the present invention may advantageously be practiced;

FIG. 3 is a schematic diagram of the sensing circuitry of the ICD IPG of FIGS. 1 and 2 for accumulating EGM peak amplitude data associated with tachyarrhythmia episodes; and

FIG. 4 is a flow chart illustrating the steps of accumulating EGM peak amplitude data associated with tachyarrhythmia episodes and subsequently employed in determination of a suitable sensing threshold and/or sense amplifier gain.

### DETAILED DESCRIPTION OF THE PREFERRED EMBODIMENTS OF THE INVENTION

In the following detailed description, references are made to illustrative embodiments of methods and apparatus for carrying out the invention. It is understood that other embodiments can be utilized without departing from the scope of the invention. In particular, the present invention is described in the context of a single chamber, ventricular ICD that provides the functions of monitoring the ventricular EGM, detecting VT, SVT, and VF, discriminating VF from VT and SVT or VT from SVT, and providing ventricular ATP regimens in response to a detected VT episode, storing data related to detected VF, VT and SVT episodes for uplink telemetry transmission to external medical devices, and optionally providing pacing for bradycardia.

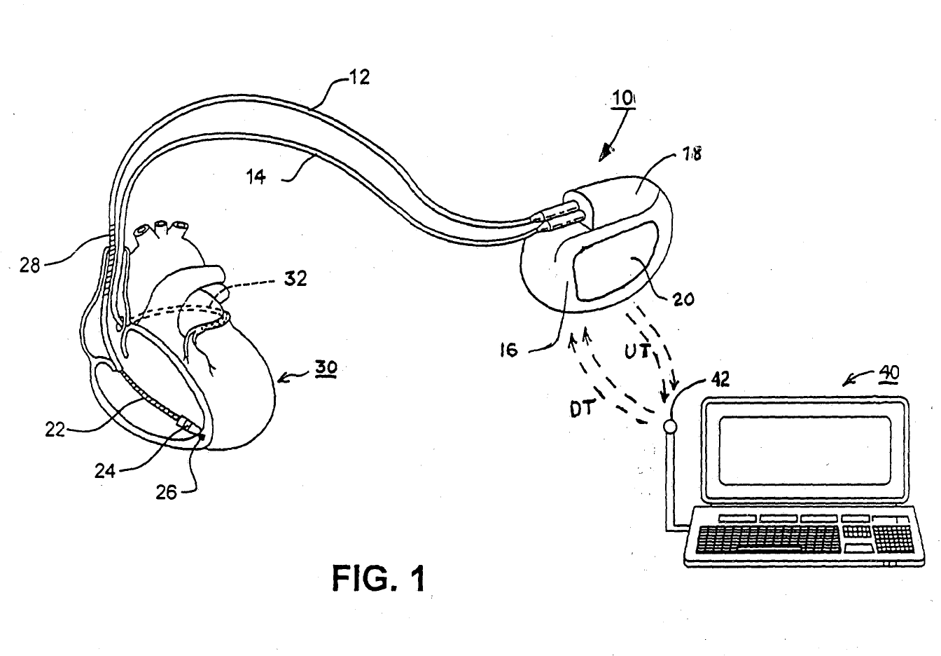

FIG. 1 illustrates one embodiment of an ICD of the type disclosed in commonly assigned U.S. Pat. No. 6,393,316, comprising an ICD implantable pulse generator (IPG) 10 in which the ATP delivery algorithms of the present invention can be advantageously incorporated and the associated ICD medical electrical leads 12 and 14 extending to a human heart 30. The ICD of FIG. 1 is also shown in relation to an external programmer 40 and external programmer telemetry antenna 42 providing uplink telemetry (UT) and downlink telemetry (DT) transmissions with an IPG antenna.

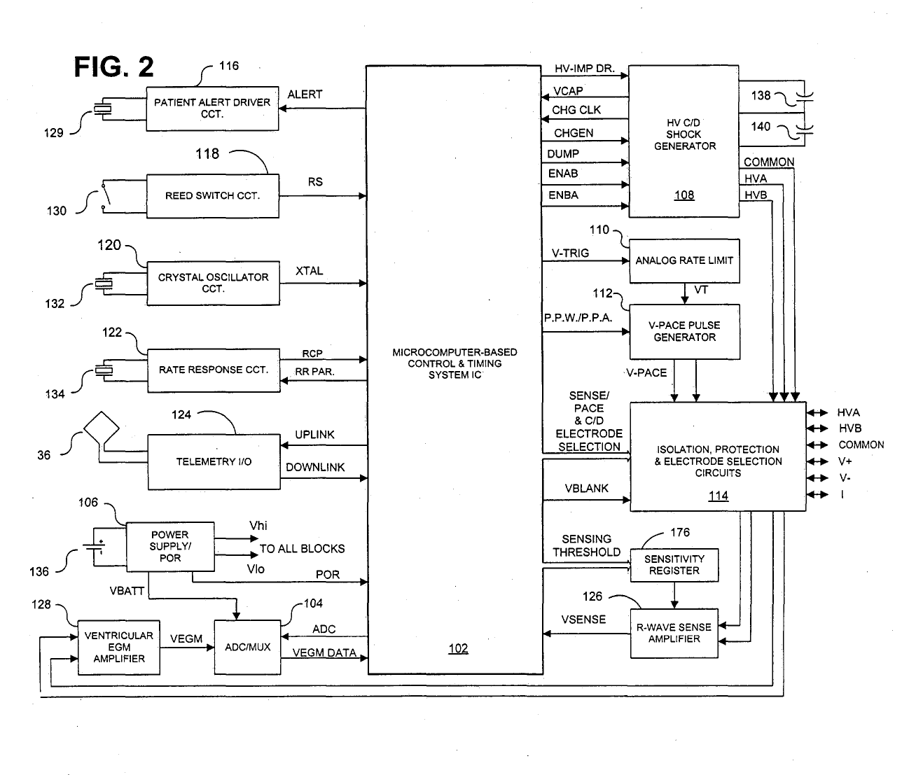

The ICD IPG 10 is formed of a hermetically sealed enclosure 16 containing the electronic circuitry and components, including a battery, depicted in FIG. 2 and a connector block 18. The proximal ends of the illustrated ICD leads 12 and 14 are inserted into two connector ports of the connector block 18 to make electrical connections between lead conductors of the ICD leads 12 and 14 and the circuitry within the hermetically sealed enclosure 16 via feedthroughs extending through the enclosure wall in a manner well known in the art. The ICD IPG 10 is intended to be implanted subcutaneously remote from the heart, and at least an uninsulated portion of the hermetically sealed enclosure 16 may be employed as an indifferent pace/sense and/or cardioversion/defibrillation electrode 20.

The ICD lead 14 is a coronary sinus (CS) lead and the ICD lead 12 is a right ventricular (RV) lead and are extended transvenously from the ICD IPG 10 into the heart chambers using conventional implantation techniques. CS lead 14 supports an elongated wire coil, cardioversion/defibrillation electrode 32 that is located in the coronary sinus and great vein region of the heart 30. The cardioversion/defibrillation electrode 32 is advanced through the coronary sinus ostium in the right atrium and around the heart, and is disposed in proximity with the left ventricular wall either in the great vein or in the coronary sinus.

The RV lead 12 supports proximal and distal, elongated wire coil, cardioversion/defibrillation electrodes 22 and 28, a ring-shaped pace/sense electrode 24, and a helical pace/sense electrode 26 comprising an active fixation helix. The helical pace/sense electrode 26 is screwed into the tissue of the right ventricle at the right ventricular apex to fix the pace/sense electrodes 24 and 26 in the right ventricle. Other RV fixation mechanisms well known in the art, e.g., soft, pliant tines, may be substituted for the active fixation helix.

The cardioversion/defibrillation electrodes 22 and 28 are disposed in the RV and superior vena cava (SVC) respectively to define one cardioversion/defibrillation vector between the base,and apex of the heart 30. An RV—LV cardioversion/defibrillation vector is defined between the cardioversion/defibrillation electrodes 22 and 32. Other cardioversion/defibrillation vectors can be defined between the subcutaneous housing electrode 20 and any of the cardioversion/defibrillation electrodes 22, 28 and 32. Pairs of the cardioversion/defibrillation electrodes 22, 28 and 32 can be selectively coupled together to define further cardioversion/defibrillation vectors in a manner known in the art.

In conjunction with the present invention, the illustrated ICD leads and described electrodes are merely exemplary of possible lead systems and electrodes that can be paired together to detect R-waves, to process the EGM, to deliver C/D shocks in response to a confirmed VF detection, and to provide pacing, particularly to the right ventricle. The illustrated ICD leads and electrodes provide a variety of sense electrodes that can be paired and coupled to a ventricular sense amplifier to detect R-waves, an EGM amplifier to sense the EGM, and to a C/D shock generator to deliver monophasic or biphasic C/D shocks to the heart to counter VF. It will be understood that other ICD leads and pace/sense and cardioversion/defibrillation electrodes can be employed in the practice of the invention as long as the electrodes provide sense electrode pairs for detection of R-waves, for sensing the EGM, and for delivering the monophasic or biphasic C/D shocks to the heart to counter VF.

For example, in the simplest case of a low cost, limited function, prophylactic ICD, the ICD leads may comprise a simpler RV lead supporting only the cardioversion/defibrillation electrode 22 and a single distal pace/sense electrode. A high-energy monophasic C/D shock can be delivered between the cardioversion/defibrillation electrode 22 and the housing cardioversion/defibrillation electrode 20. The R-waves and the EGM can be sensed between the single distal pace/sense electrode and one of the cardioversion/defibrillation electrode 22 and the subcutaneous housing electrode 20. RV pacing during bradycardia may or may not be provided between the single distal pace/sense electrode and one of the cardioversion/defibrillation electrode 22 and the subcutaneous housing electrode 20.

Returning to FIG. 1, ring electrode 24 and tip electrode 26 may be paired together and coupled to an R-wave sense amplifier to detect the occurrence of an R-wave, and ring electrode 24 and subcutaneous housing electrode 20 or one of the cardioversion/defibrillation electrodes 22, 28 and 32 may be paired together for sensing the EGM signal. Alternatively, pace/sense electrodes 24 and 26 may be used for both R-wave detection and EGM sensing. Moreover, two of the cardioversion/defibrillation electrodes 32, 22 and 28 may be paired together for sensing the EGM signal.

The ICD IPG 10 preferably comprises an ICD operating system as depicted in FIG. 2 that provides the operating modes and functions of the MEDTRONIC® GEM 7227 single chamber ICD IPG, for example, that is programmable in operating mode and parameter values and interrogatable employing the MEDTRONIC® Model 9790C external programmer 40, for example. FIG. 2 is a functional block diagram illustrating such a single chamber ICD operating system 100 that is merely exemplary of a variety of single chamber and dual chamber ICD systems having all or some of the capabilities described above in which the VT/VF discrimination functions of the present invention can be advantageously implemented.

The programming of ICD operating modes and parameters or the interrogation of data stored in the ICD IPG 10 or the initiation of UT transmission of the real time cardiac EGM is accomplished or initiated via programming or interrogation commands transmitted in a DT transmission-by programmer 40 from the external telemetry antenna 42 to an ICD telemetry antenna 36 shown in FIG. 2. In the context of the present invention, the ICD operating system stores VT/VF detection episode data and VF delivery data that can be UT transmitted to the external programmer 40 for review by a physician. The ICD IPG telemetry system decodes the commands in the DT transmission, retrieves and formats the responsive data or cardiac EGM and conveys it to the external programmer 40 as an UT transmission in any of the manners known in the art.

The ICD system 100 includes one of more ICs typically mounted on one or more hybrid circuit, a PC board mounting a number of discrete components, and further large scale, discrete components. The heart of the ICD operating system is in hardware and software in the microcomputer based timing and control system IC 102 that is coupled with the other system blocks. The system IC 102 comprises the typical components of a microcomputer with operating algorithms maintained in memory or embedded in firmware and further operating system control circuitry that is conveniently located with it. Various depicted signal and control lines interconnecting these blocks, but not all are shown for simplicity of illustration and because they play no material role in the practice of the present invention.

The large-scale, discrete, off-board, components include one or more batteries 136, HV output capacitors 138, 140, and (optionally) housing mounted, patient alert sound transducers 129 and/or activity sensors 134. The discrete components mounted to the PC board include telemetry antenna 36, reed switch 130, crystal 132, a set of HV discrete components of the HV cardioversion/defibrillation output circuitry 108, and switching and protection circuit components of block 114. These discrete components are coupled to system IC 102 through other ICs and hybrid circuits incorporating the functional blocks 104-128 and 176 described further below. A similar ICD operating system to that depicted in FIG. 2 in which the present invention can be implemented is disclosed, for example, in the above-referenced '316 patent. The depicted functional blocks and discrete components of FIG. 2 can be arranged as part of one or two LV hybrid circuits, a HV hybrid circuit and a discrete component PC board. However, it will be understood that a single hybrid circuit could be employed that incorporates and supports all of the system ICs.

The exemplary ICD operating system 100 of FIG. 2 is powered by the battery 136 coupled to power supplies in power source block 106 for developing regulated high and low voltage power supplies Vhi and Vlo that are supplied to selected ones of the other functional blocks. Preferably, battery 136 is a lithium silver vanadium battery that can be employed to provide HV capacitor charging current and that produces a voltage from about 3.2 volts when fresh to about 2.5 volts at specified end of service for a single chamber ICD and twice these values for a dual chamber ICD. Power supply 106 also includes a power-on-reset (POR) circuit that generates a POR signal initially when the battery 136 is connected with power supply 106 and any time that the voltage of battery 136 falls below a threshold voltage.

The crystal oscillator circuit 120 is coupled to clock crystal 132 and provides one or more system XTAL clock that is applied to the microcomputer-based control and timing system IC and distributed to other blocks of FIG. 2 as appropriate.

The telemetry I/O circuit 124 coupled with the IPG telemetry antenna 36 includes a UT transmitter that receives formatted UPLINK signals for uplink transmission and a DT receiver that receives and forwards DOWNLINK signals to telemetry I/O registers and control circuitry in system IC 102. In one telemetry scheme known in the art, the telemetry I/O circuit 124 is enabled to receive and decode DT interrogation and programming commands when the reed switch circuit provides the RS signal upon closure of reed switch 130 by an external programming head magnetic field. Downlink telemetry RF signals ring an L-C tank circuit including the IPG telemetry antenna 36. Other pacing functions are also affected when a magnetic field closes the reed switch 130 and the RS signal is generated in a manner well known in the art. In more recent telemetry schemes, the reed switch is not employed to receive DT transmissions, and the type of telemetry scheme employed in FIGS. 1 and 2 is not material to the present invention.

Optionally, a rate response circuit 122 is coupled to a physiologic activity sensor 134, which is preferably a transducer or accelerometer mounted to the IPG housing inner surface and provides activity correlated output signals to the rate response circuit 122 in a manner well known in the art. The rate response circuit 122 develops a rate control parameter (RCP) that is used to vary a pacing escape interval to pace the heart at a rate that provides adequate cardiac output. The signal processing of the transducer output signal by the rate response circuit 122 can be programmed through rate response parameter commands to develop the RCP in a number of ways known in the art. The RCP associated with a detected VTNF episode can also be stored in memory in the system IC 102 for UT transmission of the episode data to the external programmer 40 for analysis by the patient's attending physician.

Optionally, a patient alert driver circuit 166 is coupled to a sound emitting transducer 129, which is mounted adjacent to the interior surface of the IPG housing and is powered to emit audible warning signals in high urgency and low urgency tones to alert the patient of VF detection and imminent delivery of a C/D shock or of events or conditions of concern warranting physician intervention. The warnings that can be programmed into operation or programmed “off” include pace/sense and CV/DEFIB lead impedance out of range (too high or too low), low battery voltage, excessive charge time for charging the HV capacitors, all regimens in a programmed group of regimens exhausted for a given episode, and an indication of the number of shocks delivered in an episode”.

The block diagram of FIG. 2 depicts six input/output terminals labeled V+, V−, I, HVA, HVB, and COMMC that represent the connector terminals within the IPG connector block 104 that can be coupled to lead connector elements and lead conductors extending to the respective electrodes 24, 26, 30, 22, 32, and 28. As noted above, the number of input/output terminals and associated electrodes can be reduced to the minimal number necessary to practice the present invention.

The pace/sense isolation/protection and lead impedance switch circuits 114 selectively couple pairs of the six input/output terminals labeled V+, V−, I, HVA, HVB, and COMMC to the R-wave sense amplifier 126, the ventricular EGM amplifier 128 and the V-PACE pulse generator 112 in response to a corresponding sense/pace electrode selection command from the microcomputer-based control and timing system IC 102. The sense/pace electrode selection command is programmable by the patient's attending physician through use of the external programmer 40 as described above.

A ventricular pacing function for bradycardia operating in any of the ways that are well known in the art may or may not be included in a low cost, limited function prophylactic ICD as described above. The V-PACE generator 112 provides V-PACE pulses through the selected pace/sense electrode pair having a pulse width and pulse amplitude set by the programmed PPW/PPA commands in a WI of VVIR pacing mode. A timer in the microcomputer-based control and timing system 102 times out a programmed VVI pacing escape interval or a VVIR pacing escape interval that varies as a function of the RCP output by the rate response circuit 122. A V-TRIG signal is generated by microcomputer-based control and timing system 102 when the VVI or VVIR escape interval times out and applied to the analog rate limit circuit 110, which inhibits erroneous triggering of pacing at an unacceptably high rate in a manner well known in the art. The acceptable V-TRIG signals are passed through the analog rate limit 110 and trigger the delivery of the V-PACE pulse by the V-PACE pulse generator 112. The VVI or VVIR escape interval is restarted by a VSENSE generated by the ventricular sense amplifier 126 in response to an R-wave.

Similarly, the microcomputer-based timing and control system 102 also generates the V-TRIG signals for each V-PACE pulse of each ATP regimen that is applied to the RV in response to a detected VT episode. The plurality of V-TRIG signals are provided separated by a cycle length (CL) defining the pacing rate. The V-PACE pulses of the ATP regimen have a pulse width and pulse amplitude set by the programmed PPW/PPA commands.

The V-PACE pulse generator 112 can be coupled in response to a programming command through the pace/sense isolation/protection and lead impedance switch circuits 114 to the V+, V− input/output terminals to be thereby coupled with the pace/sense electrodes 24 and 26 to provide bipolar RV pacing. Or, the V-PACE pulse generator 112 can be coupled through the pace/sense isolation/protection and lead impedance switch circuits 114 to the V− terminal to be thereby coupled with the pace/sense electrode 26 and any of the I, HVA, HVB, and COMMC input/output terminals to be thereby coupled with the respective electrodes 20, 22, 32, and 28 to provide unipolar RV pacing.

In one preferred example, the ventricular sense amplifier 126 is coupled through the pace/sense isolation/protection and lead impedance switch circuits 114 to the V+, V− terminals to be thereby coupled with the pace/sense electrodes 24 and 26 to provide bipolar RV sensing of R-waves. The inputs to the ventricular sense amplifier 126 are disconnected from the V+, V− terminals by the pace/sense isolation/protection and lead impedance switch circuits 114 in response to and for the duration of a VBLANK signal generated by a ventricular blanking circuit in microcomputer-based control and timing system IC 102 upon delivery of a V-PACE pulse or a C/D shock.

The ventricular sense amplifier 126 comprises a programmable gain, bandpass amplifier, a threshold setting circuit, and a comparator for comparing the bandpass filtered ventricular cardiac signal amplitude to the SENSING THRESHOLD. The SENSING THRESHOLD that is provided to the ventricular sense amplifier 126 is stored in sensitivity register 176 and is programmable by the patient's attending physician through use of the external programmer 40 as described above. The ventricular sense amplifier 126 generates the VSENSE signal when it's inputs are not blanked and the amplitude of QRS complex exceeds the ventricular SENSING THRESHOLD, which is typically during the rise of the R-wave.

Similarly, the ventricular EGM (VEGM) amplifier 128 is coupled through the pace/sense isolation/protection and lead impedance switch circuits 114 in response to a programmable VEGM vector electrode selection command to a pair of the input/output terminals selected from input/output terminals V+, V−, I, HVA, HVB, and COMMC. The VEGM amplifier 128 filters and amplifies the cardiac signals and provides the VEGM signals to ADC/MUX 104. In the ADC/MUX 104, the VEGM is continually sampled at a sampling frequency of 256 Hz, and the sampled analog signal values are digitized and provided as VEGM DATA to RAM memory registers or buffers in system IC 102 for temporary storage on a FIFO basis. The temporarily stored VEGM DATA are shifted into memory registers within system IC 102 when a tachyarrhythmia episode satisfying the VT or VF rate detection and other detection criterion is detected.

VEGM DATA can be stored for retrieval in an UT transmission in memory registers to provide programmable length VEGM strips preceding and following the detection of the VTNF episode and encompassing any delivery of an ATP regimen or cardioversion/defibrillation (C/D) shock. Due to memory limitations, the stored VEGM DATA may be discarded and replaced each time a VTNF episode is detected. However, historic episode logs can be compiled and incremented in RAM in system IC 102 that provide the date, time, type of episode, cycle length, duration, and identify the last stored EGM DATA.

The depicted HV cardioversion/defibrillation output circuit 108 is of the type described in the above-incorporated '316 patent comprising a DC-DC converter and a HV output or discharge circuit for discharging the charge on the HV output capacitor bank 138 and 140 through selected ones of the cardioversion/defibrillation electrodes 22, 28, 32 and 20 of FIG. 1. The DC-DC converter comprises a HV charging circuit, a discrete HV step-up transformer, and the HV output capacitor bank 138 and 140 coupled to the secondary transformer coils. The charge on the HV output capacitor bank 138 and 140, in this case, is selectively discharged through combinations of the leads coupled with the cardioversion/defibrillation electrodes 26, 30 and 32 of FIG. 1 via HV switches in the switch circuit block 114. In a prophylactic ICD of the type described above, the depicted HV cardioversion/defibrillation output circuit 108 develops a high-energy monophasic or biphasic C/D shock that is delivered through a selected pair of the cardioversion/defibrillation electrodes 26, 30 and 32 of FIG. 1 via the HV switches in the switch circuit block 114.

The microprocessor within the microcomputer-based control and timing system 102 operates as an interrupt driven device, under control of software stored in ROM associated with microprocessor and responds to interrupts including the VSENSE output of the R-wave sense amplifier 126 and the time-out of the VVI or VVIR escape interval. Any necessary mathematical calculations to be performed by the microprocessor and any updating of the values or intervals controlled by pacer timing/control circuitry within the microcomputer-based control and timing system 102 take place following such interrupts.

In ICDs having dual chamber, atrial and ventricular, sensing capabilities, other strategies have been generally followed to detect and classify tachyarrhythmias that start with identifying atrial sensed events from P-waves and/or ventricular sensed events from R-waves and deriving atrial and/or ventricular event intervals and/or rates therefrom. In both single and dual chamber ICDs, VF has been typically detected based strictly on ventricular heart rate (or R-R interval) while VT has been detected based on ventricular heart rate along with other parameters, e.g., sudden onset, rate stability, and sensed physiological activity (exercise). For example, the R-R event intervals are compared to programmed FDI ranges and TDI ranges and to suddenness of onset criteria and rate variability criteria to distinguish various tachyarrhythmias from one another.

As described above and in the above-referenced '316 patent, the typical VT and VF detection criteria that have been employed in commercially released ICDs of the type illustrated in FIGS. 1 and 2 employ a rate/interval based timing criterion and a frequency criterion as a basic mechanism for detecting the presence of and distinguishing between ventricular tachyarrhythmias. To this end, the intrinsic ventricular heart rate is measured on a beat-to-beat basis by timing the R-R interval between successive VSENSE signals output by the R-wave sense amplifier 126. The R-R interval is compared to the interval ranges or thresholds established, typically by programming, for each of VF, fast VT, and slow VT.

The VF counter, fast VT counter, and slow VT counter function like FIFO shift registers having Y stages each set to “1” or “0” that can be implemented in hardware, firmware or software. Each time that a current R-R interval is shorter than an interval threshold, a “1”, for example, is advanced into the first stage of the register, the contents of each stage is advanced to the next stage, and the “1” or “0” in the Yth stage is discarded. Similarly, each time that a current R-R interval is longer than an interval threshold, a “0”, for example, is advanced into the first stage of the register, the contents of each stage is advanced to the next stage, and the “1” or “0” in the Yth stage is discarded. Thus, the count X of the corresponding VF counter, fast VT counter, or slow VT counter is “incremented” if a “1” is advanced into the initial stage of the register and a “0” is discarded from the Yth stage and “decremented” if a “0” is advanced into the initial stage of the register and a “1” is discarded from the Yth stage. The count X remains the same if the same bit value “1” or “0” is advanced into the initial stage of the register and is discarded from the Yth stage.

For example, the R-R interval is simultaneously compared to a programmed fibrillation detection interval (FDI), a programmed fast tachycardia interval (FTDI), and a programmed slow tachycardia detection interval (TDI). The FDI count X_{VF} is incremented if the R-R interval is shorter than the FDI and a “0” is discarded from the Yth stage or remains the same if a “0” is discarded from the Yth stage. Similarly, a slow VT count X_{VT} is incremented or remains the same in response to an R-R interval shorter than TDI but longer then the FTDI or the FDI, and a fast VT count X_{FVT} is incremented or remains the same in response to an R-R interval longer than FDI but shorter than the FTDI.

The counts X_{VF}, X_{FVT}, and X_{VT} that accumulate in the respective VF counter, fast VT counter, and slow VT counter may be used to signal detection of an associated tachyarrhythmia (VF, fast VT, or slow VT) when the count X_{VF}, X_{FVT}, or X_{VT} reaches a predetermined value referred to herein as the “number of intervals required for detection” (NID). Each rate zone may have its own defined NID, for example “VFNID” for fibrillation detection, “FVTNID” for fast VT detection and “VTNID” for slow VT detection. Thus, VF is declared when X_{VF}=VFNID, fast VT is declared when X_{FVT}=FVTNID, and slow VT is declared when X_{VT}=VTNID.

For purposes of the present invention, the particular details of implementation of the rate/interval based VT detection methodologies are not of primary importance. The present invention is practiced in the context of this exemplary ventricular ICD embodiment when VT detection criterion are met and a sequence of ATP regimens are programmed to be delivered to the RV to convert the VT to NSR. It will be understood that the present invention may also be practiced in the context of ventricular single and dual chamber anti-tachycardia pacemakers, dual chamber ICDs, and in atrial or dual chamber ICDs and atrial or dual chamber anti-tachycardia pacemakers for conversion of atrial tachycardia (AT) episodes.

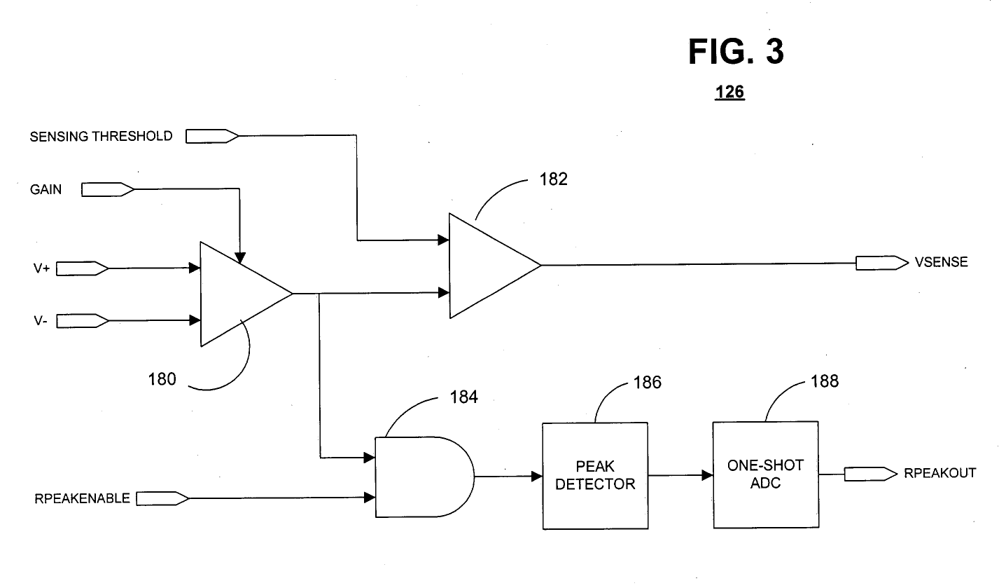

The sense amplifier circuit 126 of FIG. 2 is shown in greater detail in FIG. 3 and includes a conventional pre-amplifier stage that can be adjusted in gain by a programmed or fixed GAIN signal provided by control and timing system IC 102. The amplified ventricular signal is compared in comparator 182 to the SENSING THRESHOLD provided by sensitivity register 176. The VSENSE is output by the comparator 182 when the amplified ventricular signal reaches or exceeds the SENSING THRESHOLD. These functions and components of the sense amplifier circuit 126 can take any of the forms known in the art.

The sense amplifier circuit also comprises an R-peak detection circuit 186 that is enabled by an RPEAKENABLE signal applied to AND gate 184. The RPEAKENABLE signal is issued by circuitry in control and timing system IC 102 (when the R-R interval between successive VSENSE events meets the TDI criteria for one or more cardiac cycle or other the pre-detection criteria are met. Alternatively, the RPEAKENABLE signal can be programmed to be issued after satisfaction of any of the slow VT, fast VT and VF detection criterion. The RPEAKENABLE signal persists as long as the detection criteria are met and until delivery of a VTNF therapy. The R-peak detected by R-peak detection circuit 186 is digitized in ADC 188 and provided as the RPEAKOUT signal to the control and timing system IC 102. The ADC 188 can advantageously be a simple one-shot ADC circuit.

The RPEAKOUT signals can be temporarily stored in a buffer in control and timing system IC 102. If the RPEAKOUT signals are developed and temporarily stored starting from a detected fast interval shorter than the TDI (i.e., if a pre-detection criterion is met), then they may optionally be discarded or tagged if VTNF detection criteria are not met. The temporarily stored data set of RPEAKOUT signals are preferably stored in RAM in control and timing system IC 102 optionally time and date stamped and associated with other physiologic data and data related to the tachyarrhythmia episode and delivery of any anti-tachyarrhythmia therapies. The accumulated episode data can be stored until the IMD RAM is subsequently UT transmitted to an EMD, e.g., programmer 40. The physician can analyze the UT transmitted data in order to decide upon and program, via a DT transmitted command, a suitable SENSING THRESHOLD for the sense amplifier 126 that is normally used in detecting R-waves.

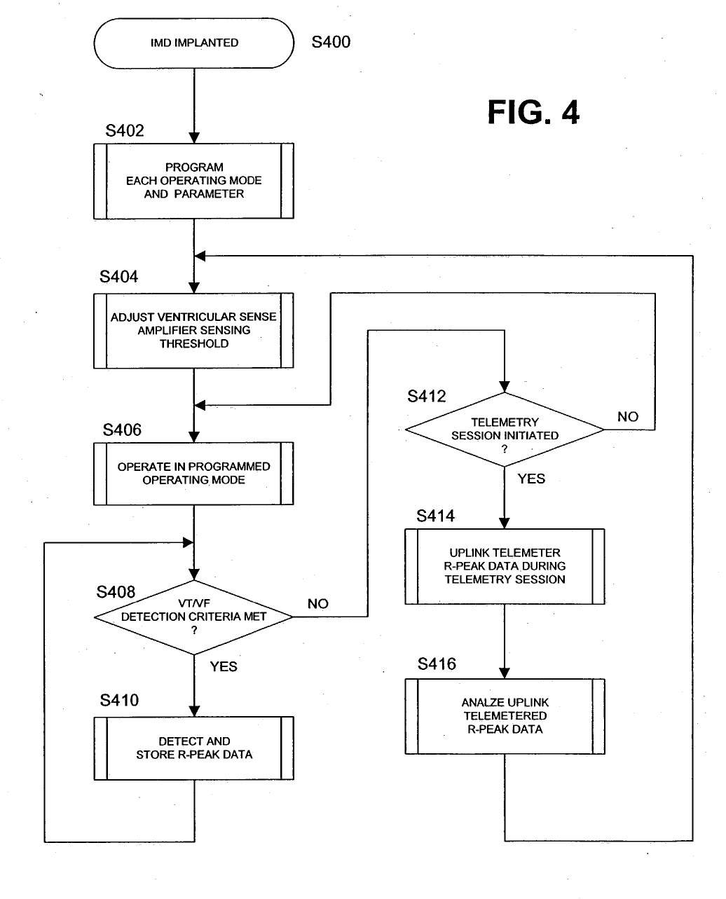

Turning to FIG. 4, the steps of accumulating EGM peak amplitude data associated with tachyarrhythmia episodes and subsequently employed in determination of a suitable SENSING THRESHOLD and/or sense amplifier gain are depicted. In step S400, the IMD, in this case the ICD 10, is implanted in a patient, and the physician programs the operating modes and parameter values, particularly the VTNF detection criteria and the VTNF therapies to be delivered, in step S402. The SENSING THRESHOLD of sense amplifier 126 of FIGS. 2 and 3 is also programmed, tested and re-programmed in step S404 until the physician is satisfied that it will adequately sense R-waves.

The ICD 10 then operates in the patient in the programmed manner in step S406. In this example, it is assumed that the R-wave peak signals are to be determined and stored in step S410 after one of the VTNF detection criteria are met in step S408. A VTNF therapy may or may not be delivered, and such steps are not illustrated to simplify the depiction.

The steps S406-S410 continue until a telemetry session is initiated as determined in step S412. The stored R-peak data is UT transmitted in step and processed by programmer 40 for analysis by the physician in step S416. Step S404 (and optionally step S402) are then repeated as deemed necessary by the physician analyzing the R-peak data in step S416.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments.

In addition, it will be understood that specifically described structures, functions and operations set forth in the above-referenced patents can be practiced in conjunction with the present invention, but they are not essential to its practice.

It is therefore to be understood, that within the scope of the appended claims, the invention may be practiced otherwise than as specifically described without actually departing from the spirit and scope of the present invention.

What is claimed is:

1. In an implantable medical device of the type including a sense amplifier that detects cardiac signals associated with intrinsic depolarizations of a heart chamber that exceed a sensing threshold for use detection of a tachyarrhythmia episode, a method comprising:
upon satisfaction of at least one pre-detection criteria associated with potential detection of a tachyarrhythmia episode, measuring the peak amplitude of the cardiac signal; and
storing one or more of the measured peak amplitude of the cardiac signal for subsequent diagnostic uses.

2. The method of claim 1, wherein a diagnostic use comprises adjusting the sensing threshold to a level related to the measured peak amplitudes to assure sensing of cardiac signals having diminished peak amplitudes during tachyarrhythmia episodes.

3. The method of claim 2, wherein the implantable medical device further includes the capability of delivering at least one anti-tachyarrhythmia therapy to the heart chamber upon satisfaction of detection criterion for a tachyarrhythmia episode.

4. The method of claim 1, wherein the implantable medical device further includes the capability of delivering at least one anti-tachyarrhythmia therapy to the heart chamber upon satisfaction of detection criterion for a tachyarrhythmia episode.

5. The method of claim 1, further comprising:
comparing the amplitude of the cardiac signal to the sensing threshold and issuing a sense event signal when the cardiac signal amplitude meets the sensing threshold; and
processing sense event signals in relation to at least one pre-detection criteria associated with potential detection of a tachyarrhythmia episode.

6. The method of claim 5, wherein a diagnostic use comprises adjusting the sensing threshold to a level related to the measured peak amplitudes to assure sensing of cardiac signals having diminished peak amplitudes during tachyarrhythmia episodes.

7. In an implantable medical device of the type that includes a sense amplifier that detects cardiac signals associated with intrinsic depolarizations of a heart chamber that exceed a sensing threshold for use detection of a tachyarrhythmia episode, a method comprising:
upon satisfaction of at least one detection criteria associated with a tachyarrhythmia episode, measuring the peak amplitude of the cardiac signal; and
storing one or more measured peak amplitude of the cardiac signal for subsequent diagnostic uses.

8. The method of claim 7, wherein a diagnostic use comprises adjusting the sensing threshold to a level related to the measured peak amplitudes to assure sensing of cardiac signals having diminished peak amplitudes during tachyarrhythmia episodes.

9. The method of claim 8, wherein the implantable medical device further includes the capability of delivering at least one anti-tachyarrhythmia therapy to the heart chamber upon satisfaction of the at least one detection criteria for a tachyarrhythmia episode.

10. The method of claim 7, wherein the implantable medical device further includes the capability of delivering at least one anti-tachyarrhythmia therapy to the heart chamber upon satisfaction of the at least one detection criteria for a tachyarrhythmia episode.

11. The method of claim 10, further comprising:
comparing the amplitude of the cardiac signal to the sensing threshold and issuing a sense event signal when the cardiac signal amplitude meets the sensing threshold; and
processing sense event signals in relation to at least one detection criterion associated with potential detection of a tachyarrhythmia episode.

12. The method of claim 11, wherein a diagnostic use comprises adjusting the sensing threshold to a level related to the measured peak amplitudes to assure sensing of cardiac signals having diminished peak amplitudes during tachyarrhythmia episodes.

13. In an implantable medical device of the type that includes a sense amplifier that detects cardiac signals associated with intrinsic depolarizations of a heart chamber that exceed a sensing threshold for use detection of a tachyarrhythmia episode, a system comprising:
means for measuring the peak amplitude of the cardiac signal upon satisfaction of at least one pre-detection criteria associated with potential detection of a tachyarrhythmia episode; and
means for storing one or more measured peak amplitude of the cardiac signal for subsequent diagnostic uses.

14. The system of claim 13, wherein a diagnostic use comprises adjusting the sensing threshold to a level related to the measured peak amplitudes to assure sensing of cardiac signals having diminished peak amplitudes during tachyarrhythmia episodes.

15. The system of claim 14, wherein the implantable medical device further includes the capability of delivering at least one anti-tachyarrhythmia therapy to the heart chamber upon satisfaction of detection criterion for a tachyarrhythmia episode.

16. The system of claim 13, wherein the implantable medical device further comprises means for delivering at least one anti-tachyarrhythmia therapy to the heart chamber upon satisfaction of detection criterion for a tachyarrhythmia episode.

17. The system of claim 13, further comprising:
means for comparing the amplitude of the cardiac signal to the sensing threshold and issuing a sense event signal when the cardiac signal amplitude meets the sensing threshold; and
means for processing sense event signals in relation to at least one pre-detection criteria associated with potential detection of a tachyarrhythmia episode.

18. The system of claim 17, wherein a diagnostic use comprises adjusting the sensing threshold to a level related to the measured peak amplitudes to assure sensing of cardiac signals having diminished peak amplitudes during tachyarrhythmia episodes.

19. In an implantable medical device of the type that includes a sense amplifier that detects cardiac signals associated with intrinsic depolarizations of a heart chamber that exceed a sensing threshold for use detection of a tachyarrhythmia episode, a system comprising:
means for measuring the peak amplitude of the cardiac signal upon satisfaction of at least one detection criteria associated with a tachyarrhythmia episode; and
means for storing one or more measured peak amplitude of the cardiac signal for subsequent diagnostic uses.

20. The system of claim 19, wherein a diagnostic use comprises adjusting the sensing threshold to a level related to the measured peak amplitudes to assure sensing of cardiac signals having diminished peak amplitudes during tachyarrhythmia episodes.

21. The system of claim 20, wherein the implantable medical device further comprises means for delivering at least one anti-tachyarrhythmia therapy to the heart chamber upon satisfaction of the at least one detection criteria for a tachyarrhythmia episode.

22. The system of claim 19, wherein the implantable medical device further comprises means for delivering at least one anti-tachyarrhythmia therapy to the heart chamber upon satisfaction of the at least one detection criteria for a tachyarrhythmia episode.

23. The system of claim 22, further comprising:
means for comparing the amplitude of the cardiac signal to the sensing threshold and issuing a sense event signal when the cardiac signal amplitude meets the sensing threshold; and
means for processing sense event signals in relation to at least one detection criterion associated with potential detection of a tachyarrhythmia episode.

24. The system of claim 23, wherein a diagnostic use comprises adjusting the sensing threshold to a level related to the measured peak amplitudes to assure sensing of cardiac signals having diminished peak amplitudes during tachyarrhythmia episodes.
